(19) United States
(12) Patent Application Publication
(10) Pub. No.: US 2023/0203421 A1
(43) Pub. Date: Jun. 29, 2023

(54) LOW INTENSITY VIBRATION SYSTEM AND METHOD FOR BIOPROCESSING

(71) Applicant: THE RESEARCH FOUNDATION FOR THE STATE UNIVERSITY OF NEW YORK, Albany, NY (US)

(72) Inventors: Mei Lin CHAN, Huntington Station, NY (US); Clinton T. RUBIN, Port Jefferson, NY (US)

(73) Assignee: THE RESEARCH FOUNDATION FOR THE STATE UNIVERSITY OF NEW YORK, Albany, NY (US)

(21) Appl. No.: 17/928,483

(22) PCT Filed: May 28, 2021
(86) PCT No.: PCT/US2021/034742

§ 371 Date: Nov. 29, 2022

Related U.S. Application Data
(60) Provisional application No. 63/031,743, filed on May 29, 2020.

(51) Int. Cl. C12M 3/06; B01F 35/22; B01F 35/222; B01F 31/24

(52) U.S. Cl. CPC C12M 27/16; B01F 31/24; B01F 35/222; B01F 35/2202; B01F 2215/0454

(57) ABSTRACT

The present disclosure is directed to devices, systems and methods that include a stage and an actuator configured to transmit a orthogonal force to the stage, wherein the actuator is configured to receive a plurality of orthogonal acceleration signals, wherein the orthogonal acceleration signals comprise an actuator frequency signal and an actuator magnitude signal.

### CROSS REFERENCE TO RELATED APPLICATIONS

The present application claims the benefit of priority from U.S. Provisional Application No. 63/031,743, filed on May 29, 2020, the content of which is incorporated herein by reference in its entirety.

### FIELD OF THE DISCLOSURE

The present disclosure relates to devices and methods for use of low intensity vibrations in the promotion of cell growth of a variety of cell types. The device can include a moving/oscillating platform or stage, upon which any suitable size container can be supported, with that container containing one or more types of cell, in suspension or adhered to a surface, for proliferation.

One aspect of this disclosure is to apply Low Intensity Vibration (LIV) to enhance proliferation of cells, which is a volume scalable and time scalable method. This approach can enhance cell growth outcomes by using mechanical signals in the form of LIV to stimulate proliferation and protein production in such cells.

### BACKGROUND OF THE DISCLOSURE

Many biomanufacturing processes depend on the use of living biological cells cultured in bioreactors. These cells are used to produce biomaterials and therapeutic biomolecules, as well as boost host T cell numbers for personalized medicine. Current and future biomanufacturing applications include, but are not limited to, treatment of cancer, enhancement of the immune system, combatting infectious diseases, ameliorating metabolic dysfunction, and building bioactive scaffolds for tissue engineering and regeneration.

As demand for biomanufacturing products continues to increase; optimization strategies are of interest to improve yields in large-scale commercial production of therapeutic proteins, or more quickly expand cell numbers for personalized medicine applications such as autologous immunotherapy using CAR-T cells for blood cancer treatments. Traditionally, optimization schemes involve invasive cell line development via vector genetic engineering, cell engineering or omics-based approaches to modulate and ultimately improve the transcriptional activities. In addition, culture media formulation and chemical environment may be modulated, while protein purification and characterization cultivated post-processing.

While cell-based biomanufacturers believe advances that improve proliferation or production yields by as little as 10% would be transformational, the industry remains hesitant to explore or introduce processes that require biological or chemical modifications or violate sterile systems by introducing outside agents.

Thus, what is desired is a system, devices, and methods to enhance cell proliferation in a variety of cell types. Embodiments of the present disclosure provide devices and methods that address the above needs.

### SUMMARY OF THE DISCLOSURE

The present disclosure is directed to devices, systems and methods that include a stage and an actuator configured to transmit a force to the stage, wherein the actuator is configured to receive a plurality of acceleration signals, wherein the acceleration signals comprise an actuator frequency signal and an actuator magnitude signal.

### BRIEF DESCRIPTION OF THE DRAWINGS

The patent or application file contains at least one drawing executed in color. Copies of this patent or patent application publication with color drawings will be provided by the office upon request and payment of the necessary fee.

The present disclosure will be better understood by reference to the following drawings, which are provided as illustrative of certain embodiments of the subject application, and not meant to limit the scope of the present disclosure.

FIG. 1 is an illustration of one embodiment of a device of the present disclosure.

FIG. 2A is a graphical illustration of orthogonal acceleration signals.

FIG. 2B is a block diagram of a system for controlling an actuator of the device, according to one embodiment of the disclosure.

FIGS. 2C and 2D are a diagram illustrating a high-level flow for controlling an actuator of the device, according to an embodiment of the disclosure.

FIG. 2E is a flow diagram illustrating training and operation of a machine learning model, according to an embodiment of the disclosure.

FIG. 2F is a block diagram of another system for controlling an actuator of the device, according to an embodiment of the disclosure.

FIG. 3 is a graphical illustration of the Linker of Nucleoskeleton and Cytoskeleton (LINC) complex coupling the nucleus to the actin cytoskeleton through connectivity of Nesprin with Sun at the inner nuclear membrane, as dependent on the Klarsicht, ANC-1, Syne Homolog (KASH) domain.

FIGS. 4a-4d are graphical illustrations of the long-term effects of LIV on cell proliferation. FIG. 4(a) is a graphical illustration of long term-LIV improved proliferation over 30 w, adding up to 81% (p<0.001) cumulative difference of cell doubling at passage 60 (P60). FIG. 4(b) is a graphical illustration of proliferation, which was concomitant to increased CyclinA2 (Ccna-2) expression at P17 (p<0.05, n=3/grp). FIG. 4(c) is a graphical illustration of the relationship between cumulative doubling and passage number, specifically between passages 15 and 25, LIV did not augment proliferation in MSCs that overexpress dominant negative Nesprin KASH plasmid that de-functionalizes LINC complex. FIG. 4(d) is a graphical illustration of LIV treated wild-type or empty plasmid expressing MSCs showing 57% and 51% increase cell proliferation, respectively (p<0.05, n=8/grp), there was no benefit of LIV in KASH plasmid expressing MSCs.

FIGS. 5a-5b are graphical illustrations of proliferative responses to LIV. LIV stimulation with identical intensity, frequency, duration, and refractory period (0.2 g, 500 Hz, 3×30 mins/d, 2 h refractory period) induced significant but contrary cell proliferation responses in (FIG. 5a) CHO-adherent cells (+79%) and (FIG. 5b) CHO-suspension cells (-13%) (n=6, mean ± SD, p≤0.05).

FIGS. 6a-6b are graphical illustrations of proliferative responses to LIV. When subjected to same LIV protocol (30 Hz, 0.7 g, 2 bouts of lh/d, 2-hour refractory period), while (FIG. 6a) CHO-Suspension cells resulted in a 210% increase in cell proliferation, while decreasing proliferation in hybridoma cells by 24%. *p<0.05 (FIG. 6b). Data presented as mean ± SD.

FIGS. 7a-7b are graphical illustrations of proliferative responses to LIV. (FIG. 7a) Altering LIV Hz as applied to CHO-suspension cells showed a 61% increase at 30 Hz, 27% more than 90 Hz. (FIG. 7b) Then, altering intensity at 30 Hz, 0.7 g was identified as more influential than other inputs. (n=6, mean ± SD, p≤0.05).

FIGS. 8a-8b are graphical illustrations of the affect varying LIV signals have on cells. FIG. 8a a 30 Hz, 0.7 g, 2×30 mins/day, 2 h refractory period, increased proliferation in CD3 Pan T cell by 24.8% compared to controls (n=5, ± SD, p≤0.05 for significance). FIG. 8b increasing from 1 to 2 LIV doses elevated proliferation by 31% (p<0.05), while 3 doses increased proliferation by 39% over sham (p=0.01).

FIGS. 9a-9c are graphical illustrations of the affect varying LIV signals have on cells. In FIG. 9a CD4+ and in FIG. 9b CD8+ T cells are subset of CD3+ Pan T cells commonly used for CAR-T immunotherapy to treat lymphoma. LIV-induced increased proliferation of Pan T cells promoted significant increase in CD4+ (18.6%) and CD8+ (22.6%) T cells, without altering either migratory function nor activation (FIG. 9c) as indicated by CD62L and CD25 biomarkers, respectively. (n=6, mean ± SD, p≤0.05 for significance).

FIG. 10 is a graphical illustration of the scalability of cell proliferation. LIV signals were applied to CD3+ Pan T cells in T75 flasks (0.7 g, 30 Hz, 2× 1 h bouts/d, 3 h refractory period), increased proliferation by 47% at 5 days (n=6, mean ± SD, p≤0.05), suggesting clinically relevant populations of T cells can be reached sooner.

FIGS. 11a-11f represent data from full or partially filled T75 flasks subject to LIV signals and fluid motions. FIG. 11a LIV @ 90 Hz and 0.7 g, generating peak motion of 20 µm. FIG. 11b peak displacement via camera frames between t=0 and t=π/2 determined acceleration transmittance and sloshing. FIG. 11c vectors were parallel to actuator motion with a maximum of 18 pm, 90% of the 20 µm actuator motion and FIG. 11d minimal sloshing (<2 µm). FIG. 11e, peak motion of partially filled flasks was >2.5 mm - two orders larger than filled, similar to that measured from top view FIG. 11f.

FIG. 12 includes a graphical illustration and images demonstrating the impact of low magnitude mechanical signals (LMMS), delivered using LIV, activated pFAK (left & center), while LIV-induced RhoA activation was prevented when FAK inhibitor PF was present (right). S.E. p<0.01.

FIG. 13 are images demonstrating actin impact, as compared to control (left), LIV induces perinuclear actin remodeling (right), evident 1h after vibration treatment by actin bundling, including bridging over. Scale bar = 15 µm.

FIG. 14 includes a graphical illustration and images demonstrating the impact of LMMS, while LMMS elevated FAK, a second brief bout after a 2 h rest period doubled LMMS-induced pFAK production (left), resulting in more mature FAs. S.E., ***<0.01.

FIG. 15 is a photograph of one embodiment of a device of the present disclosure with a plurality of containers supported by the stage.

FIG. 16 is a graphical illustration of a control wave, showing high fidelity Z-axis vertical acceleration.

FIGS. 17A-17D are graphical illustrations of the viability and function of CD4+ T cells that have or have not received an LIV signal.

FIGS. 18A-18D are graphical illustrations of the viability and function of Pan T cells that have or have not received an LIV signal.

### DETAILED DESCRIPTION OF THE DISCLOSURE

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein.

As used herein, the term “substantially”, or “substantial”, is a broad term and is used in its ordinary sense, including, without limitation, being largely but not necessarily wholly that which is specified, which is equally applicable when used in a negative connotation to refer to the complete or near complete lack of an action, characteristic, property, state, structure, item, or result. For example, a surface that is “substantially” flat would mean either completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper”, “lower”, “right”, “left”, “vertical”, “horizontal”, “top”, “bottom”, “orthogonal” and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element and a second element are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

As used herein, the term “actuator” is understood to mean a device capable of changing a dimension (e.g., extending or compressing its length) in response to a control signal (e.g., an electrical signal). Certain (but not all) types of actuators may include a movable element that moves in a first direction (e.g., upwards), relative to an actuator housing, during extension of the actuator and in a second direction (e.g., downwards), relative to the actuator housing, during compression of the actuator. In certain implementations, an actuator may be capable of exerting an acceleration and a force on the movable element in the direction of motion of the movable element, thereby actively facilitating said motion. In certain implementations, an actuator (e.g., an electro-hydraulic actuator) may also be capable of exerting an acceleration and a force on the movable element opposite the direction of motion of the movable element. In certain implementations, an actuator may be capable of exerting an acceleration and a force on the movable element even in the absence of motion of the movable element. In certain implementations, an actuator may function as a passive or semi-active damper. In certain implementations, an actuator may be capable of operating in at least three quadrants of a force-velocity diagram. In certain implementations, an actuator may be capable of operating in all four quadrants of a force-velocity diagram. An electro-hydraulic actuator is understood to mean an actuator that includes an electric motor, a hydraulic pump, and the movable element (e.g., a piston). Other types of actuators may include an electromechanical actuator (e.g., a ball screw), an electrical actuator (e.g. a linear motor), and a speaker. As used herein, the term “speaker” can, for example, refer to a device such as an electroacoustic transducer that can convert an electrical signal to an output such as sound and/or pressure waves, which are capable of delivering a frequency and magnitude of acceleration signal over a range of frequencies and magnitudes.

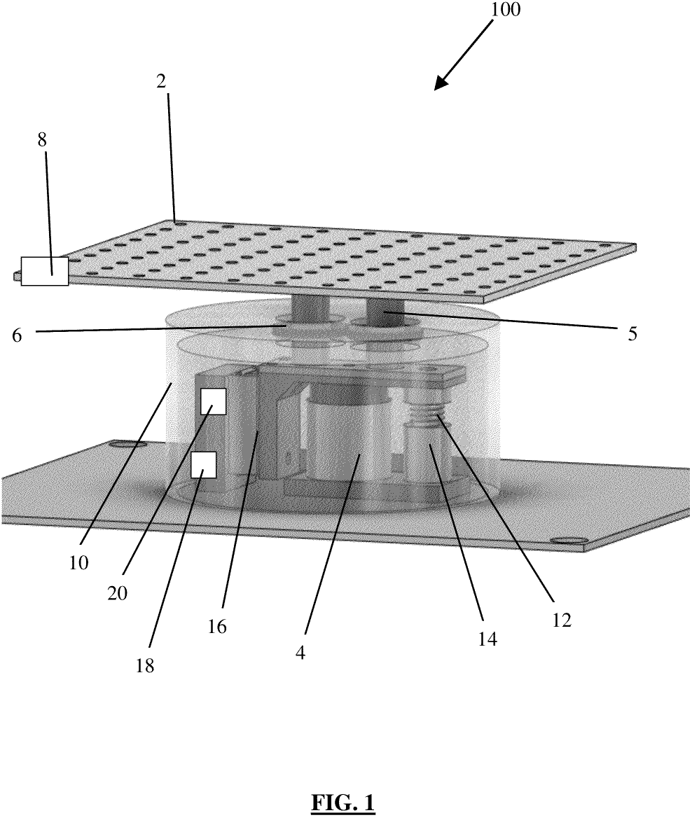

FIG. 1 is an illustration of one embodiment of a device 100 of the present disclosure. The device 100 comprises a stage 2, which is operably connected to an actuator 4. In some embodiments, one actuator 4 is included, in other embodiments at least one actuator 4 can be present, such as two actuators, three, four, five, tens or hundreds of actuators. The stage 2 can be any suitable size, shape (such as substantially planar and/or substantially curved such as a bowl) is configured to support a container, which itself is configured to maintain a volume of liquid.

Alternatively, the stage itself can be a portion of a vessel or container such as any suitably sized tank. In this embodiment, the actuator is effectively connected to the vessel or container itself. In alternative embodiments, one, two or more actuators can be connected to the vessel or container in any suitable pattern and/or matrix. Further, in this alternative embodiment, the two or more actuators can receive a coordinated set of orthogonal acceleration signals, with each individual orthogonal acceleration signal discussed below.

The container, and the device 100 itself, can be of any sufficient size. For example, the stage 2 of the device 100 can be configured to support a container with a volume of about 1 mL or less, about 1 mL or more, or about 50 mL or more, or about 100 mL or more, or about 500 mL or more, or about 1 L or more, or about 2.5 L or more, or about 5 L or more, or about 10 L or more, or about 25 L or more, or about 50 L or more, or about 100 L or more, or about 250 L or more, or about 500 L or more, or about 1,000 L or more, or about 5,000 L or more, or about 10,000 L or more. The container can be any suitable object that is capable of maintaining a volume of liquid. The container may be formed of any suitable material, such as plastic, glass, ceramic, metal, carbon-based materials, rubber, and combinations thereof, and can be rigid, substantially rigid, and/or flexible such as a bag.

The actuator 4 is configured to transmit an acceleration and a force, which includes an orthogonal acceleration and force, to the stage 2 according to a received plurality of orthogonal acceleration signals. As used herein, the term “orthogonal” can refer to an angle that is perpendicular, substantially perpendicular, or within about 25°, or more or less, of being perpendicular to an axis of movement of the actuator 4. As shown in the figures the orthogonal acceleration is provided substantially vertically to a plane of a container, however, in other embodiments, the orthogonal acceleration can be directed to any portion of a container or vessel, such as, for example, substantially horizontally through a side wall of the container or vessel.

These orthogonal acceleration signals can be received from an external processor through any suitable wired and/or wireless connection (such as Bluetooth, Wi-Fi, Near Field Communication, etc.), and/or from a processor 18 within the device 100.

The processor 18 (in this sentence and throughout the discussion, “processor” refers to processor 18 and/or an external processor from which the actuator 4 can receive the plurality of orthogonal acceleration signals) can be a CPU. The processor 18 can be a single core or a multiple core processor. In other aspects, some of the processor 18 can be a graphics processing unit (GPU). In other aspects, the processor 18 can be an integrated circuit, application specific integrated circuit (ASIC), programmable logic device (PLD), digital signal processor (DSP), field programmable gate array (FPGA), logic gate, register, semiconductor device, chip, microchips, chip set, and so forth. When multiple processors 18 are used, the different processors can each be of a different type. For example, one processor may be a CPU and another processor may be a GPU or an ASIC. In other embodiments using multiple processors 18, each of the multiple processors can be the same type of processor such as, for example, two or more CPU’s.

Device 100 can also include an electronic storage device/memory 20. The electronic storage device (as well as any database referred to herein) can include computer system readable media in the form of volatile memory, such as random access memory (RAM) and/or cache memory or others. In some embodiments, multiple electronic storage devices may be used. The electronic storage device can be any type of integrated circuit or other storage device adapted for storing data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

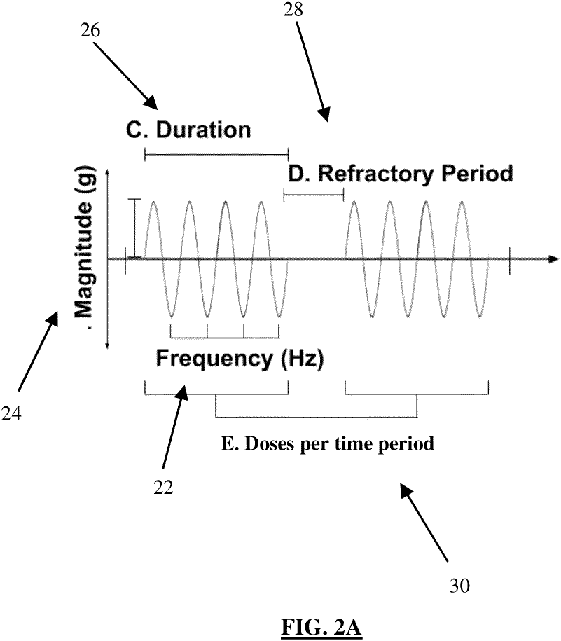

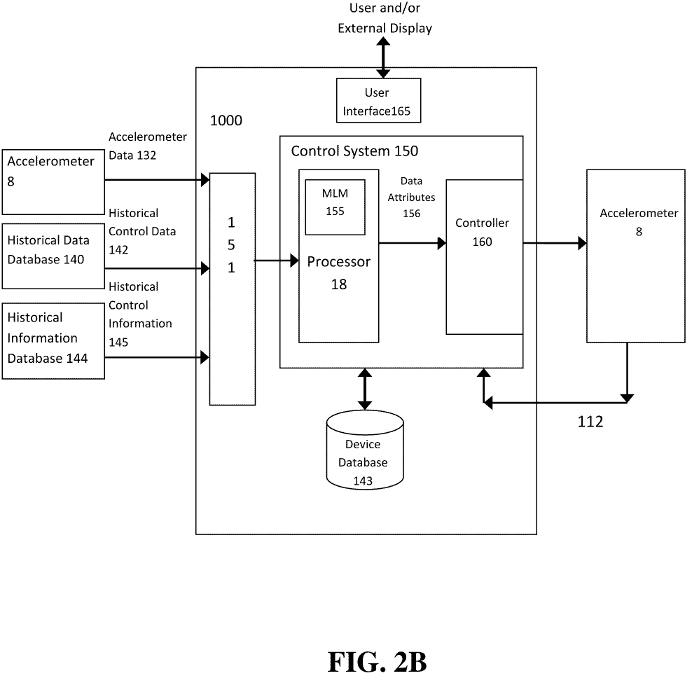

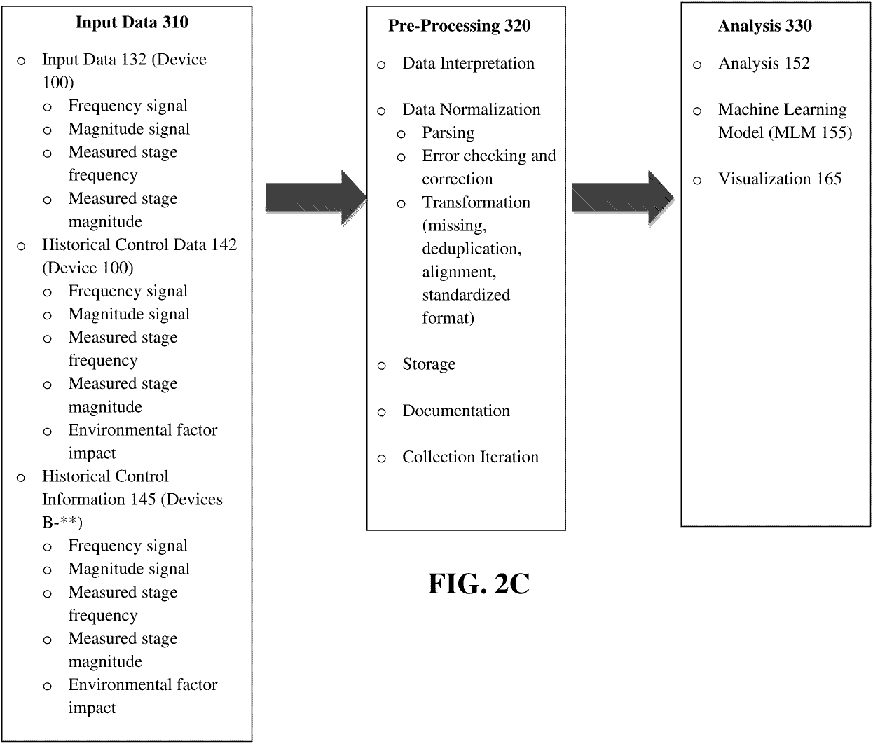

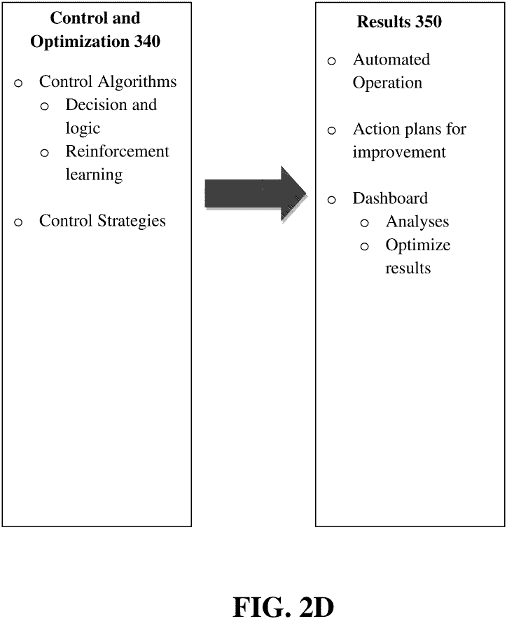

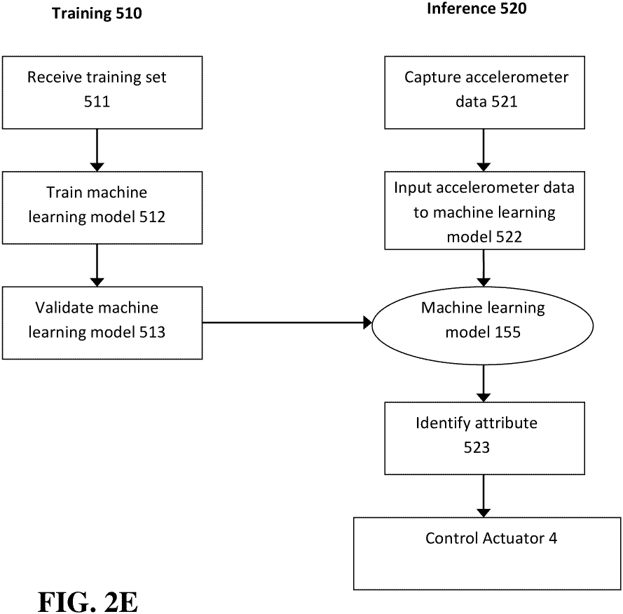

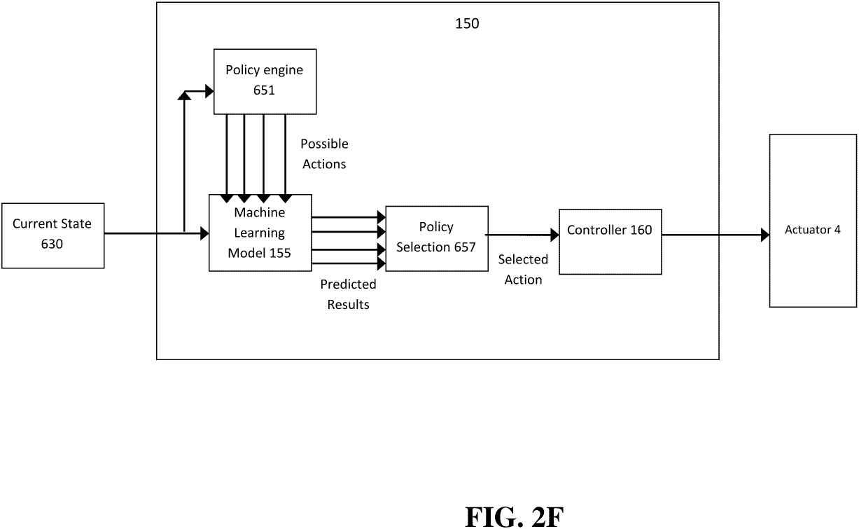

The actuator 4 is configured to receive the plurality of orthogonal acceleration signals from the processor 18 and/or an external processor. These orthogonal acceleration signals can comprise a multitude of different signals, such as a frequency signal and a magnitude signal, which are discussed further in reference to FIG. 2A below.

Also, optionally included in the device 100 are bellows 6, which are configured to allow movement to be transmitted from the actuator 4, through an actuator arm 5, which passes through a portion of a case 10 of the device 100, to the stage 2, with the bellows 6 creating a substantially airtight seal between an interior of the case 10 and the stage 2. The actuator arm 5 is operably attached at a first end to the actuator 4, and operably attached at a second end to the stage 2. The case 10 is shown as being substantially transparent in FIG. 1, however in other embodiments, the case 10 can be of any suitable opacity, and can be formed of any suitable material, such as plastic, glass, ceramic, metal, carbon-based materials, rubber, and combinations thereof.

An optional slider 16 can be operably attached to any portion of the actuator 4 and/or the actuator arm 5, with the slider 16 acting to reduce friction between elements, such as movement between the actuator arm 5 and the case 10.

An optional elastic element 12, such as a helical spring, is configured to bring the actuator 4 into substantial equilibrium during operation. An optional elastic element cover 14 is configured to extend around at least a portion of the elastic element 12 and can be configured to fix the elastic element 12 to the case 10.

An optional accelerometer 8 can be operably attached to any portion of the stage 2 and/or the actuator arm 5. As used herein, the term “accelerometer” includes any type of accelerometer, vibration sensor, or instrument that can be used to measure acceleration, vibration, and/or movement, including but not limited to an accelerometer, gyroscope, magnetometer, or any similar instruments. As used herein the “accelerometer” is configured to provide an electrical signal representative of such measured movement, in any direction, as a measurement, such as a measure of sound wave, a measure of a vibration, a measure of acceleration, and/or a measure of a motion. The accelerometer 8 can comprise a transducer for converting an acoustic signal into an electromagnetic signal. In some embodiments, a transducer is connected to the accelerometer 8.

As used herein, the term electrical “signal” refers to a varying quantity that can carry information, such as a sound wave, an acoustic signal, an electromagnetic signal, such as an electromagnetic signal produced by an accelerometer in response to detecting any frequency range of signal. As used herein, the term “signal processing” refers to a field of techniques used to extract information from signals.

The accelerometer 8 is configured to transmit the measured signal (a stage frequency signal and/or a stage magnitude signal) to the processor 18 and/or an external processor through any suitable wired and/or wireless data transfer operation. Further, the accelerometer 8 can comprise a suitable cover, such that the accelerometer 8 is substantially separated from moisture one or near the stage 2.

The actuator 4 is configured to receive the plurality of orthogonal acceleration signals from the processor 18 and/or an external processor. These orthogonal acceleration signals can comprise a multitude of different signals, such as a frequency signal and a magnitude signal, which are discussed further in reference to FIG. 2A below.

FIG. 2A is an example of various signals and is referred to for explanatory purposes. As can be seen in FIG. 2A, the orthogonal acceleration signals (LIV signals) configured to be received by the actuator 4, and transmitted to the stage 2 through actuator arm 5, can include one or more of a frequency signal 22 (in Hertz (Hz)), a magnitude signal 24 (in gravitational force(G)), a duration signal 26 (in any suitable time period), a refractory period signal 28(in any suitable time period between periods of duration) and a doses per time signal 30(in any suitable time period durations of signal per unit time, for example per day).

As used herein, the term “acceleration” in orthogonal acceleration signals refers to the rate of change of the velocity of the stage 2, and/or the rate of change of the velocity of the accelerometer 8, and/or the rate of change of the velocity of a container supported by the stage 2, and/or the rate of change of the velocity of the contents of the container supported by the stage 2- each caused by movement of the actuator 4. Further, as used herein, the term “acceleration” may refer to positive acceleration, and may also refer to negative acceleration, which may sometimes be referred to a deceleration.

The frequency signal can be a “low intensity vibration” (LIV) signal. As used herein, the term LIV signal comprises the frequency signal 22 and the magnitude signal 24. The frequency signal 22 can be between about 0.1 Hz to about 1,000 Hz, or about 10 Hz to about 500 Hz, or about 20 Hz to about 150 Hz, or about 30 Hz to about 90 Hz, or about 30 Hz to about 35 Hz. The magnitude signal 24 can be about 2 G’s or less, about 1.5 G’s or less, about 1.4 G’s or less, about 1.3 G’s or less, about 1.2 G’s or less, about 1.1 G’s or less, about 1 G or less, about 0.9 G’s or less, about 0.8 G’s or less, about 0.7 G’s or less, about 0.6 G’s or less, about 0.5 G’s or less, about 0.4 G’s or less, about 0.3 G’s or less, about 0.2 G’s or less, or about 0.1 G’s or less.

The duration signal 26 can be any suitable duration for the actuator 4 to transmit the LIV signal, such as about 1 minute, about 2 minutes, about 5 minutes, about 10 minutes, about 15 minutes, about 20 minutes, about 30 minutes, about 45 minutes, about 1 hour, about 90 minutes, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, or more.

The refractory period (refractory signal 28) can be any suitable duration between periods of the actuator transmitting the LIV signal, such as about 1 minute, about 2 minutes, about 5 minutes, about 10 minutes, about 15 minutes, about 20 minutes, about 30 minutes, about 45 minutes, about 1 hour, about 90 minutes, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, or more.

The doses per time signal 30 (sessions/periods of LIV signal delivery) can be any suitable number of doses over any suitable length of time, such as once per about one week, once per about 3 days, once per about 2 days, once per about 24 hours, once per about 20 hours, once per about 16 hours, once per about 12 hours, once per about 8 hours, once per about 6 hours, once per about 5 hours, once per about 4 hours, once per about 3 hours, once per about 2 hours, once per about one hour, twice per about one hour, three times per about one hour, four timer per about one hour, or more.

The LIV signal can optionally include a carrier signal of any suitable frequency and magnitude. The waveform of the LIV signal can be one or more of a substantially sinusoidal wave, a substantially compound signal, a substantially square wave, a substantially triangular wave, a substantially sawtooth wave, and combinations thereof.

The measured signal of the accelerometer 8, which is a measured, stage frequency signal and/or a measured, stage magnitude signal, can be transmitted to the processor 18 and/or an external processor through any suitable wired and/or wireless data transfer operation. The processor can then compare the measured, stage frequency signal to the frequency signal 22. The processor can also compare the measured, stage magnitude signal to the magnitude signal 24.

To accurately control the stage and any contents it supports, the difference between either or both of (1) the measured, stage frequency signal and the frequency signal 22 and (2) the measured, stage magnitude signal and the magnitude signal 24 should be substantially small (e.g., within about 1%, about 2%, about 5%, about 10%, about 15%, about 20%, etc.). In such a case, no modification of the frequency signal 22 and/or the magnitude signal 24 is taken.

However, due to various circumstances, if the difference between either or both of (1) the measured, stage frequency signal and the frequency signal 22 and (2) the measured, stage magnitude signal and the magnitude signal 24 is sufficiently large, thus above the threshold, one or both of the frequency signal 22 and the magnitude signal 24 can be updated/modified to bring the difference between either or both of (1) the measured, stage frequency signal and the frequency signal 22 and (2) the measured, stage magnitude signal and the magnitude signal 24 to below the threshold.

This update/modification can occur in a stepwise fashion to either automatically, serially (if needed) increase or automatically, serially (if needed) decrease the frequency signal 22 to an updated frequency signal and/or magnitude signal 24 to an updated magnitude signal until either or both of (1) the measured, stage frequency signal and the frequency signal 22 and (2) the measured, stage magnitude signal and the magnitude signal 24 are below the threshold.

This update/modification can also/alternatively occur based on inputting either or both of (1) the measured, stage frequency signal and (2) the measured, stage magnitude signal to a machine learning model that predicts whether or not, and to what degree, a change to either or both of (1) the frequency signal 22 and (2) the magnitude signal 24 needs to be made to bring the difference between either or both of (1) the measured, stage frequency signal and the frequency signal 22 and (2) the measured, stage magnitude signal and the magnitude signal 24 to below the threshold value.

FIG. 2B is a block diagram of a system 1,000 for controlling the actuator 4 of device 100, according to one embodiment of the present disclosure. The actuator 4 is configured to maintain, decrease, or increase one or both of the frequency signal 22, and the magnitude signal 24.

The system 1,000 includes a data interface 151 and control system 150. The control system includes a processor (which may be processor 18 as shown in FIG. 2B, or a processor outside of the device 100), which includes a machine learning model (MLM) 155, and a controller 160. As used herein, the term “machine learning model” is meant to include a single machine learning model or an ensemble of machine learning models. Each model in the ensemble may be trained to infer different attributes. The MLM 155 is a program module of the processor (18) that performs the methods and functions described herein. The MLM 155 can be programmed into the integrated circuits of the processor (18).

The data interface 151 receives various input data, which are processed, at least in part by the machine learning model (MLM 155). The results output by the MLM 155 are data attributes 156, which are received as input by the controller 160, which controls the actuator 4 accordingly. Although FIG. 2B illustrates the system 1,000 being separate from the device 100, the system 1,000 can be incorporated within a housing and/or structure of the device 100, or the system 1,000 can transmit signals through any wired or wireless transmission modality.

The control system 150 can receive various types of inputs, and from various sources. This includes accelerometer data 132, captured by the accelerometer 8, historical control data 142 received from their storage locations in a historical data database 140, and historical information 145 received from their storage locations in a historical information database 144.

The accelerometer data 132 captured by the accelerometer 8 includes (1) the measured, stage frequency signal 22′ and (2) the measured, stage magnitude signal 24′. The accelerometer 8 can capture data continuously, or it can be configured to capture data every “N” seconds or “N” minutes.

The historical control data 142 received from storage locations in the historical data database 140 can be any previously captured data by the accelerometer 8 for a specific container and specific liquid amount. This previously captured data can be recent, for example, from the past several hours or days, or this previously captured data can be more distant, for example from the past several weeks or months.

The historical information 145 received from storage locations in the historical information database 144 can be previously captured data by the accelerometer 8 of the device 100, or from other accelerometers on similar devices. This historical information database 144 can include all captured data from all devices (device 100 and other same/similar devices) that are similarly situated such as, for example, supporting the same number/size of containers. This previously captured data in the historical information database 144 can be recent, for example, from the past several hours or days, or this previously captured data can be more distant, for example, from the past several weeks or months. This previously captured data in the historical information database 144 can also come from other locations.

Historical data database 140 and historical information database 144 can be local databases within system 1,000 and/or within the structure of the device 100. One or both of the historical data database 140 and historical information database 144 can be a remote or cloud-based database, which is accessible by system 1,000 through a network connection.

In FIG. 2B, the control system 150 also receives information of (1) the measured, stage frequency signal 22′ and (2) the measured, stage magnitude signal 24′ from the accelerometer 8 as data 112. The data 112 of (1) the measured, stage frequency signal 22′ and (2) the measured, stage magnitude signal 24′ can be used by the system 1,000 to perform further comparisons between a desired frequency and signal (received by the actuator 4) and (1) the measured, stage frequency signal 22′ and (2) the measured, stage magnitude signal 24′, so further signal adjustments to the actuator 4 can be made.

Device database 143 contains profile information for the actuator 4 and can also contain historical profile information of a number and/or size of container supported by the stage 2 the actuator 4 moves. This profile information can include frequency and magnitude capabilities of the specific actuator 4 under various load conditions based on the load of containers supported by the stage 2 the actuator 4 is connected to.

The control system 150 receives these different data, processes them in the processor (18) them and controls, with the controller 160 the accelerometer 8 accordingly.

The MLM 155 can be useful to predict data attributes that can be difficult or cumbersome to develop using more conventional approaches. For example, the accelerometer data 132 may be used as input to the MLM 155, which then predicts various data attributes 156. The controller 160 then controls the actuator 4 according to these attributes. One example is that the MLM 155 may predict an increase of 10% of the frequency signal 22 to the actuator 4 so that the measured, stage frequency signal 22′ is within the difference threshold of the frequency signal 22.

The use of machine learning is especially beneficial for situations where the predicted attribute is a complex function of two or more factors, or when there is a desire for the system to self-learn or self-monitor certain relationships. For example, if the accelerometer data132 indicates that an increase in the frequency signal 22 and a decrease in the magnitude signal 24 is needed to be within the difference thresholds. Machine learning approaches can be used to learn these complex relationships for each specific stage 2 and each specific load of containers. In addition, these complex relationships may change over time as the actuator 4 degrades, or environmental attributes such as temperature, humidity, barometric pressure etc. impact actuator 4 operability. Even if it were possible to expressly construct a model to regulate an actuator, it is desirable for machine learning techniques to automatically adapt to changes over time rather than manually changing the model to account for these shifts.

Returning to FIG. 2B, the system 1,000 also includes an optional user interface 165. The user interface 165 provides an interface to the system 1,000, allowing an operator to monitor in real-time the actuator 4 and/or accelerometer data 132, and/or to review historical performance and/or to predict future performance. Through the user interface 165, the operator can also make changes to the device database 143. It may also allow the operator to configure different data inputs 132, 142, 145. Alternatively, or in addition to, the user interface 165 can transmit viewable data to an external display, such as one on a housing of the device 100, or an external display not physically connected to the device 100, such as a separate monitor and/or a mobile electronic device such as a mobile phone or tablet.

Alternatively, or in addition to, the user interface 165 can transmit an alert signal to an external server. This alert signal can be based on accelerometer data 132 indicating a dangerous and/or important condition such as rapid changes in frequency and/or magnitude, which may indicate that a load of containers has been added to the stage 2 or a load of containers has been removed or fallen from the stage 2.

This external server can be configured to receive such an alert signal, and then automatically transmit it to one or more mobile devices such as, for example, a mobile device of a researcher, engineer and/or operator of the device 100.

FIGS. 2C and 2D are a diagram illustrating a high-level flow for controlling the actuator 4, according to one embodiment of the disclosure. FIG. 2D is a continuation of FIG. 2C. Whereas FIG. 2B illustrates control concepts in the form of a system block diagram, FIGS. 2C and 2D organizes these concepts as a flow of data, actions, and results. The input data 310 in FIG. 2C correspond to the inputs to the control system 150 in FIG. 2B. The input data 310 includes sensor data 132 (which can include one or more of device 100′s’s frequency signal 22, magnitude signal 24, measured stage frequency 22′ and measured stage magnitude 24′), historical control data 142(which can include historical measurements from one or more of device 100′s’s frequency signal 22, magnitude signal 24, measured stage frequency 22′, measured stage magnitude 24′ and historical environmental factor impact), and historical control information 145 (which can include historical measurements from one or more other devices, and their historical frequency signal, magnitude signal, measured stage frequency, measured stage magnitude and historical environmental factor impact). FIG. 2C lists examples of each of these categories, which are also described with respect to FIG. 2B.

The input data 310 is pre-processed 320. This can include data interpretation and data normalization. Examples of normalization include, for example, parsing data, error checking and correction, and transformation. Missing data may be retrieved or noted as missing. Duplicate data may be de-duplicated or “de-duped”, i.e., duplicate data points are removed. Data from different sources may be aligned in time or space. Data may be reformatted to standardized formats used in further processing. Pre-processing 320 may also include data storage (e.g., in a memory of the system 1,000 and/or an external database), documentation and collection iteration. Documentation is the process of documenting the context of data, collection methodology, structure, organization, descriptions of variables and metadata elements, codes, acronyms, formats, software used, access and use conditions, and the like. Collection iteration is the process of iteratively collecting new forms of data and/or improving previous data collection procedures to improve data quality.

Pre-processed data is analyzed 330. Analytics performed by processor (18) can be performed for purposes of controlling the actuator 4 or for purposes of analyzing the actuator 4. Analysis can identify various patterns, as well as identifying areas of waste or potential improvement. As described above, MLM 155 is especially useful to learn complex relationships and/or to automatically adapt to changes.

Visualization of analysis results can be presented by the user interface 165. This user interface 165 can be configured to display various data and responses over time, as well as, for example, error alerts.

Continuing to FIG. 2D, based on the analysis 330, different types of control and optimization 340 can be implemented. For more traditional control algorithms, the control is defined by a set of control logic or rules. Reinforcement learning can be used to adapt control strategies over time. FIG. 2D also lists some specific control strategies, such as automatic increases of frequency and/or magnitude signal received by the actuator 4 above a stepwise increase. Control and optimization may be performed based on machine learning results. For example, how large the automatic increase of frequency and/or magnitude signal should be based on variations of input data 132 and previous variations and responses in historical control data 142 may be learned through machine learning analysis.

Box 350 lists some of the results and benefits that may be achieved. Improved control can result in avoidance of operation outside frequency and magnitude thresholds. Automatic discovery of patterns and adaptation can result in a more automated operation of the actuator 4. It may also be useful to produce a dashboard that gives an overview of operation of the actuator 4.

FIG. 2E is a flow diagram illustrating training and operation of a machine learning model (MLM 155), according to an embodiment. The process includes two main phases: training 510 the MLM 155 and inference (operation) 520 of the MLM 155. These will be illustrated using an example where the machine learning model learns to predict the required increase in frequency signal the actuator 4 is to receive based on historical data of increases in frequency signal and their impact on measured, stage frequency. The following example will use the term “machine learning model” but it should be understood that this is meant to also include an ensemble of machine learning models.

A training module (not shown) performs training 510 of the MLM 155. In some embodiments, the MLM 155 is defined by an architecture with a certain number of layers and nodes, with biases and weighted connections (parameters) between the nodes. During training 510, the training module determines the values of parameters (e.g., weights and biases) of the MLM 155, based on a set of training samples.

The training module receives 511 a training set for training the machine learning model in a supervised manner. Training sets typically are historical data sets of inputs and corresponding responses by other actuators in other devices. The training set samples the operation of the actuator 4, preferably under a wide range of different conditions. FIG. 2C gives some examples of input data 310 that may be used for a training set. The corresponding responses are alterations of the actuator 4 and subsequent observations after some time interval, such as the measured, stage frequency after an increase in frequency signal 22.

The following is an example of a training sample at 12:00 PM:
i. Frequency signal of 30 Hz
ii. Measured, stage frequency 25 Hz

At 12:00 PM, the actuator 4 receives an updated actuator frequency signal from the processor (18) to increase the frequency signal 10%.

At 12:05 PM the observed responses, measured by the accelerometer 8 are the following:
i. Frequency signal of 33 Hz
ii. Measured, stage frequency 28 Hz

Since the frequency threshold in this example is 1.5 Hz, and the desired frequency is 30 Hz, the actuator 4 can then receive another updated actuator frequency signal from the processor (18) to increase the frequency signal 5% to 34.65 Hz.

At 12:10 PM the observed responses, measured by the accelerometer 8 are the following:
i. Frequency signal of 34.65 Hz
ii. Measured, stage frequency 29.4 Hz

Since the frequency threshold in this example is 1.5 Hz, and the desired frequency is 30 Hz, the stage is operating within the threshold and the actuator 4 does not need to receive another updated actuator frequency signal from the processor (18).

In this example, the increase in the frequency initially of 10% is not sufficient to bring the measured, stage frequency to a level within the threshold. Only after a total increase of 15.5% from the initial frequency signal will the accelerometer 8 be measuring the measured, stage frequency within the threshold limits. The MLM 155 can use this as an example to learn that if the frequency signal (and desired stage frequency) received by the actuator 4 is 30, but the initial, measured, stage frequency is 25 Hz, an immediate increase of 15.5% will bring the measured, stage frequency to within the acceptable threshold.

In typical training 510, a training sample is presented as an input to the MLM 155, which then predicts an output for a particular attribute. The difference between the machine learning model’s output and the known good output is used by the training module to adjust the values of the parameters (e.g., features, loads, or biases) in the MLM 155. This is repeated for many different training samples to improve the performance of the MLM 155 until the deviation between prediction and actual response is sufficiently reduced.

The training module typically also validates 513 the trained MLM 155 based on additional validation samples. The validation samples are applied to quantify the accuracy of the MLM 155. The validation sample set includes additional samples of inputs, known responses from the actuator 4, and subsequent accelerometer 8 measurements. The output of the MLM 155 can be compared to the known ground truth. To evaluate the quality of the machine learning model, different types of metrics can be used depending on the type of the model and response.

Classification refers to predicting what something is, for example if an image in a video feed is a person. To evaluate classification models, F1 score may be used. The F1 score is a measure of predictive accuracy of a machine learning model. The F1 score is calculated from the precision and recall of the machine learning model, where the precision is the number of correctly identified positive results divided by the number of all positive results, including those not identified correctly, and the recall is the number of correctly identified positive results divided by the number of all samples that should have been identified as positive.

Regression often refers to predicting quantity, for example, how much energy is consumed. To evaluate regression models, coefficient of determination, which is a statistical measure of how well the regression predictions approximate the real data points, may be used. However, these are merely examples. Other metrics can also be used. In one embodiment, the training module trains the machine learning model until the occurrence of a stopping condition, such as the metric indicating that the model is sufficiently accurate or that a number of training rounds having taken place.

Training 510 of the MLM 155 can occur off-line, as part of the initial development and deployment of system 1,000. Under this option, training samples from other devices similar to device 100 (historical control information 145) can be used to train the MLM 155. This training data can be all available historical control information 145, or a portion of the historical control information 145 for other devices that are similarly situated such as, for example, having the same/similarly capable actuator, and having the same/similar load of containers on their respective stages.

The trained MLM 155 is then deployed in the field. Once deployed, the MLM 155 can be continually trained 510 or updated. For example, the training module uses data captured in the field, during use of the actuator 4, to further train the MLM 155. The training 510 can occur within control system 150 and/or in an external database.

In operation 520, the MLM 155 uses the same inputs as input 522 to the MLM 155. The MLM 155 then predicts the corresponding response. In one approach, the MLM 155 calculates 523 a probability of possible different outcomes, for example the probability that a measured, stage frequency will be within a threshold of a desired frequency after an increase in frequency signal 22, received by the actuator 4, of 10%. Based on the calculated probabilities, the MLM 155 identifies 523 which attribute is most likely. In a situation where there is not a clear cut winner, the MLM 155 may identify multiple attributes and ask an operator of the device 100, or a third party, to verify.

FIG. 2F is a block diagram of the control system 150 that uses the MLM 155 to evaluate different possible courses of action. In this example, the MLM 155 functions as a simulation of the device 100. The current state 630 of the device’s frequency and magnitude signals received by the actuator 4, and the measured, stage measurements of the frequency and magnitude, measured by the accelerometer 8 are the inputs to the MLM 155. The control system 150 can take different courses of action to affect the frequency and magnitude, measured by the accelerometer 8. For example, the control system 150 can increase/decrease the frequency signal 22, and/or increase/decrease the magnitude signal 24 received by the actuator.

A “policy” is a set of actions performed by the control system 150. The policies can be a set of logic and rules determined by domain experts. They can also be learned by the control system 150 itself using reinforcement learning techniques. At each time step, the control system 150 evaluates the possible actions that it can take and chooses the action that maximizes evaluation metrics. It does so by simulating the possible subsequent states that may occur as a result of the current action taken, then evaluates how valuable it is to be in those subsequent states.

Based on the current state 630, a policy engine 651 determines which polices might be applicable to the current state. This can be done using a rules-based approach, for example. The MLM 155 predicts the result of each policy. The different results are evaluated and a course of action is selected 657 and then carried out by the controller 160. A set of metrics is used to evaluate the policies. Policy selection 657 can also be reported to one or both of the historical data database 140 and the historical information database 144 in any suitable way, so that historical policies can be referred to and used.

To simulate subsequent states, the control system 150 uses the trained MLM 155. When underlying conditions (e.g., environmental conditions, load of containers on the stage 2) are changing, the MLM 155 can make predictions on what most likely will be observed as a result of actions taken. Based on these predictions, the control system 150 chooses a policy or action that most likely maximizes the metric of interest, being the measured, stage frequency and magnitude continuing to be within acceptable limits of the frequency signal 22 and the magnitude signal 24.

To decide which action to take from a state, the control system 150 may employ techniques of exploitation and exploration. Exploitation refers to utilizing known information. For example, a past sample shows that under certain conditions, a particular action was taken, and good results were achieved. The control system 150 may choose to exploit this information and repeat this action if current conditions are similar to that of the past sample. Exploration refers to trying unexplored actions. With a pre-defined probability, the control system 150 may choose to try a new action. For example, 10% of the time, the control system may perform an action that it has not tried before but that may potentially achieve better results.

A further element that can be added to device 100 is one or more containers, which can be supported by the stage 2. The one or more containers may rest on the stage 2, or be operably attached such as through an adhesive or strap. Alternatively, the device 100 can be attached to a portion of a large vessel through any suitable connection means.

Examples of several containers on stage 2 are shown in FIG. 15, and are further referenced below. These containers can be any suitable size, such as a well of an assay plate, containing 1 mL or less, or a flask containing tens milliliters, to bottles containing one, two, ten or more Liters, to large vessels containing hundreds or thousands of Liters.

The container(s) itself can be of any suitable material, such as plastic, glass, ceramic, metal, organic material, carbon based material, and combinations thereof. The container(s) can be any suitable shape that is capable of storing a liquid, in which liquid a plurality of cells can be present. These plurality of cells include any suitable cells that can proliferate in a suitable liquid. For example, these plurality of cells can be one or more of bacteria, yeast cells, plant cells and animal cells. Specifically, the plurality of cells can be selected from mammal stem cells, T cells and combinations thereof. The mammal can be any suitable mammal, such as a human, cat, dog, rat, mouse, ape, etc.

Even more specifically, the mammal stem cells can be mesenchymal stem cells (MSCs). Also, more specifically, the T cells can be selected from the group consisting of CD4+ T cells, CD8+ T cells, and CD3+ Pan T cells.

Any of the cells can be adhered to a surface within the container, suspended in the liquid within the container, or a combination of adhered to a surface and suspended.

As used herein, the term “suspend” or “suspended” is meant to refer to individual cells or aggregates of cells can move throughout the liquid volume and/or rest on a surface of the container without having a substantial link or bond to the container.

As used herein, the term “adhere” or “adhered” is meant to refer to ionic, covalent, electrostatic, or other chemical attachment of a cell to a surface within the container. The surface can be an internal surface of the container itself, a surface of an internal extension, which extends from an internal surface of the container, and/or a particle within the container.

As used herein, the term “particle” refers to beads and other microscopic objects of similar size (e.g., from about 0.1 to 120 microns, or from about 1 micron to 50 microns) or smaller (e.g., from about 0.1 microns to 150 nm). For example, the term “particle” further encompasses objects of similar size (e.g., from about 0.1 microns to about 5,000 nm) of any suitable shape and dimension.

Portions of the surface of the container can be substantially flat or planar, such that it is substantially two dimensional. Alternatively, or in addition to the substantially flat or planar portions, the surface of the container can be roughened, grooved, include extensions therefrom, etc., such that the surface is substantially three dimensional.

As noted above, the actuator 4 is configured to receive the plurality of orthogonal acceleration signals from the processor 18 and/or an external processor, specifically the frequency signal 22 and the magnitude signal 24. However, the actuator 4 can also receive the duration signal 26, the refractory period signal 28, and the doses per time signal 30. Each of these signals can be manually input to the processor 18 by a user, so that the processor 18 can transmit the static signal to the actuator 4, or the processor 18 can receive further information and use that information to modify any of the frequency signal 22, the magnitude signal 24, the duration signal 26, the refractory period signal 28, and the doses per time signal 30. Such further information can be information from a flow cytometer, or other cell measurement apparatus, that can determine the concentration/number of cells in the liquid of the container at any time during the administration of LIV signals. This determined number can be compared, by the processor 18, to a theoretical value or a value of known previous uses of the device 100.

The processor 18 can then, if there is a difference between the measurement and the theoretical/known value, the processor 18 can then modify (increase or decrease) any one of the frequency signal 22, the magnitude signal 24, the duration signal 26, the refractory period signal 28, and the doses per time signal 30 in order to bring the measured value to within a threshold of the theoretical/known value.

This update/modification can occur in a stepwise fashion to either automatically, serially (if needed) increase or automatically, serially (if needed) decrease any of any one of the frequency signal 22, the magnitude signal 24, the duration signal 26, the refractory period signal 28, and the doses per time signal 30 until a cell measurement is within the threshold.

This update/modification can also/alternatively occur based on inputting the measured cell value into a machine learning model that predicts whether or not, and to what degree, a change to any of the frequency signal 22, the magnitude signal 24, the duration signal 26, the refractory period signal 28, and the doses per time signal 30 is to be taken to be within the cell count threshold.

The machine learning model of system 1,000 discussed above can be used for this purpose in addition to, or alternatively, the use described in FIGS. 2B-2F, to modify the actuator 4 based on readings from the accelerometer 8. Thus, in other embodiments, cell count values can be input as data 132 in the model noted above, and the output of that model can be whether or not, and to what degree, a change to any of the frequency signal 22, the magnitude signal 24, the duration signal 26, the refractory period signal 28, and the doses per time signal 30 is to be taken.

The processor 18 can then transmit an updated frequency signal 22′, updated magnitude signal 24′, updated duration signal 26′, updated refractory period signal 28′, and/or updated doses per time signal 30′ to the actuator 4.

As an additional feature of the device 100, it can be an element of a system which can include the processor 18, or an external processor.

The disclosure is further explained in the following examples, which are provided for illustrative, not limiting, purposes.

### EXAMPLES

### Perceiving Mechanical Signals at the Cellular Level

Many or all cells (bacteria, yeast cells, plant cells, animal cells, etc.) are sensitive to mechanical signals. Cellular response can be proportional to a product of duration and magnitude of the signal, with even brief exposure (< 15 minutes) to an LIV signal generating a response.

The capacity of LIV to bias mesenchymal stem cell (MSC) fate selection towards osteoblastogenesis and away from adipogenesis can be augmented by the incorporation of a rest period (refractory period/refractory signal 28), such as a pause of about 3 hours before applying another LIV signal.

### Role of LINC in Transmitting Mechanical Signals to the Nucleus

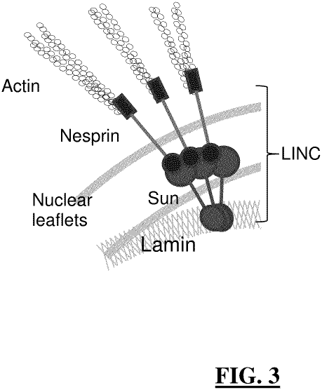

Exogenous mechanical signals are transduced to the nucleus through a system of cytoskeletal components (e.g., focal adhesions) to control differentiation and function. Cytoplasmic actin is coupled to laminA/C in the nucleus through Linker of Nucleoskeleton and Cytoskeleton (LINC) complex proteins. F-actin binds to giant nesprin, a spectrin repeat protein that pierces the nuclear envelope, connecting via its “KASH” (Klarsicht, ANC-1, Syne Homology) domain to intra-membrane leaflet sun proteins, as illustrated graphically in FIG. 3.

Mechanical signals are transduced from the cytoskeleton across the nuclear envelope via the LINC connection. Nuclear-cytoskeletal tethering provided by the LINC complex is important for the mechanosensory function of the nucleus. As such, Nesprin4-^{/-} and Sun1^{-/-} mice are both rendered deaf due to mechanical isolation of nucleus, attenuating any sensation of sound vibrations.

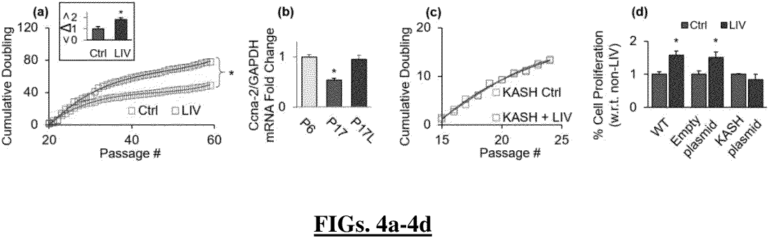

Such a mechanosensory phenotype is consistent with findings that LINC connectivity is required for LIV induced signaling and may promote sensitivity of cells to mechanical signals such as those disclosed herein. Disruption of LINC via knock down of sun proteins 1 & 2 (siSUN) decreased MSC mechanosensitivity and accelerated adipogenesis, as illustrated in FIGS. 4a-4d.

The laminA/C network provides a scaffolding network to sun proteins that extend into the inner nuclear leaflet, and provide a direct transmission of mechanical signals, and reinforce the premise that LINC dysfunction would mechanically isolate the nucleus, preventing mechanoresponse at the level of gene expression. As such, promoting LINC connectivity via tuned mechanical signals may enhance biomanufacturing endpoints.

### Disruption of LINC Suppresses Mechanosensitivity

LIV is a mechanical regime modeled after physiologic, high frequency muscle contractions, and in MSCs, LIV promotes proliferation. To test the long-term effect of LIV on cell proliferation in vitro, primary MSCs were subjected to a twice-daily LIV regimen, which has been shown to be an effective driver of MSC proliferation and differentiation. As seen in FIG. 4a, at passage 30 (P30), proliferation rates of non-LIV cells (i.e., sham controls) begin to fall; in contrast, cultures exposed to LIV maintained an 81% average increase of cell doubling at P60.

Sustained proliferation was reflected by increased CyclinA2 expression at P17, as illustrated in FIG. 4b. When LINC complex function was disabled by a dominant negative form of the Nesprin KASH domain (pCDH-EF1-MSC1-puro-mCherry-Nesprin-laKASH, Jan Lammerding, Cornell), the salutary effect of LIV on proliferation was reduced significantly, as illustrated in FIGS. 4c and 4d. FIGS. 4a-4d support that LIV-induced recovery of MSC proliferation under simulated microgravity includes functional LINC complexes.

### Improving Efficacy of Biomanufacturing With LIV

For therapeutic protein production, recombinant Chinese hamster ovary (CHO) cells are the most commonly used mammalian expression systems for large-scale production of recombinant protein. Examples include human monoclonal antibody (mAb) for treating cancer, human bone morphogenic protein-2 (hBMP-2) for treating osteoporosis, erythropoietin for treating anemia, and human growth hormone (hGH) for improving cell reproduction and regeneration. For tissue engineering, regeneration, and immunotherapy, it is important to control the expansion rate of differentiated cells such as adipocytes, chondrocytes, osteoblasts and immune cells such as macrophages and T cells. Other than influencing the proliferation rate, the ability to bias the differentiation pathway of stem cells could expand tissue engineering strategies and applications.

In considering mechanical inputs, studies have reported on the negative impact of fluid-induced shear stress on CHO cells. For example, in terms of recombinant protein production from CHO cells, a study shows that cell specific hGH production decreased from 100% at 0.005 N m⁻² to 49% at 0.80 N m⁻² relative to the no-shear control. Notably, published studies show an inhibitory rather than positive impact of shear stress on biomanufacturing.

In contrast, the present disclosure demonstrates that both adherent and suspension CHO cells are mechanosensitive, responding to LIV signals with significant increased expansion rate and protein production. As discussed herein, CHO adherent cells and suspension cells respond differently to the same LIV signal parameters. Varying the LIV signal through

Variation of the frequency signal 22, the magnitude signal 24, the duration signal 26, the refractory period signal 28, and the doses per time signal 30 enables the ability to administer a “custom” LIV signal for a specific cell type to optimize desired cellular responses.

### Influence of LIV on Proliferation in CHO Adherent and Suspension Cells

Proliferation outcome is signal specific, with what promotes proliferation in one cell type can hamper it in another. For example, while both adherent and suspension CHO cells were responsive to an LIV signal, they responded to the LIV signal very differently, with adherent cells increasing 79% to a 500 Hz, three dose, 30 minute 0.2 g dosing (FIG. 5a), while the same signal suppressed proliferation in CHO suspension cells by 13% (FIG. 5b).

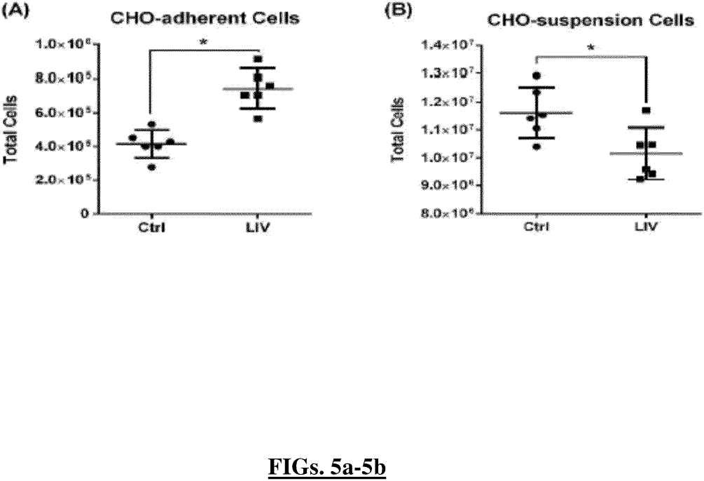

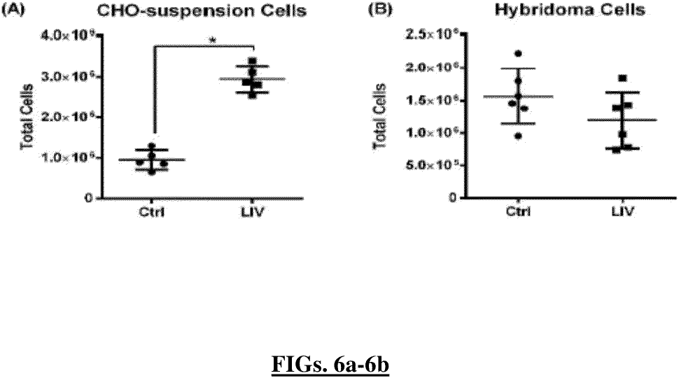

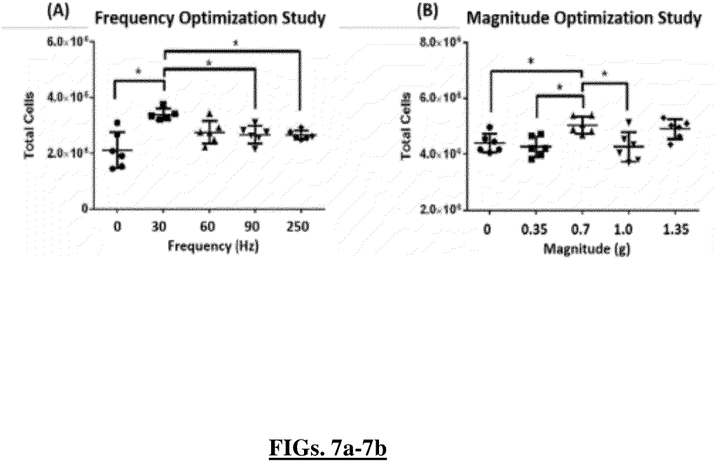

Comparing CHO-suspension to hybridoma cells, stimulated by a 30 Hz, 0.7 g, with two 1h doses, increased proliferation three-fold in CHO-suspension (FIG. 6a), but this same signal caused a non-significant reduction in the hybridoma cells (FIG. 6b). While the variables of an ‘optimal’ LIV signal are many, a fractional factorial design can be used, with a first emphasis on frequency, and intensity. For example, by first identifying 30 Hz as an influential frequency in CHO-suspension cells (61% over sham control; FIG. 7a), it was determined that a 0.7 g intensity elicited the greatest proliferative response (FIG. 7b).

It has been shown that mechanical input is delivered across – and can autotregulate – the cell’s cytoskeleton, but this adaptation is transient, resorting back to ‘unstimlulated’ within about 24 h. In the case of the adherent cell, this involves both generation and rearrangement of focal adhesions and their attachment to nucleus. In non-adherent cells, the cytoskeleton is released from plasma membrane attachment to substrate – but as cells move through, for example, the vascular system, the nucleus is pushed and pulled through tight spaces maintaining a specific spatial context within the cell as determined by cytoskeletal contractions. LIV signals, in the absence of substrate contact, can also alter the perinuclear cytoskeleton.

### LIV Signal Affect on Human T Cells

Increasing the rate of proliferation of human T cells could be used to shorten the expansion phase of autologous cell therapy (e.g., CAR-T biomanufacturing), thus reducing the time required to grow sufficient numbers for treatment. Results herein are indicative that both intensity and frequency of sinusoidal LIV signals can be adjusted to promote proliferation of human Pan T cells (FIG. 8a). While these cells mostly were not affected with 10 Hz & 90 Hz LIV signals, a 30 Hz signal stimulated a 27% increase in proliferation over controls.

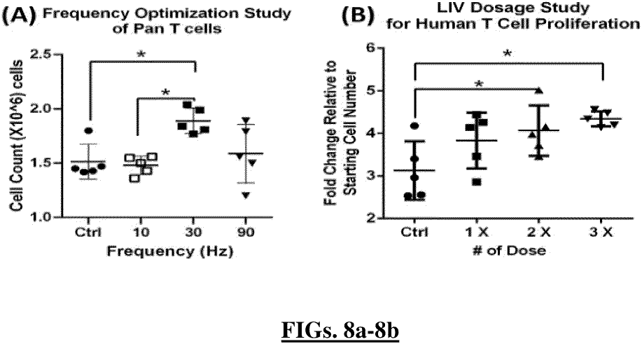

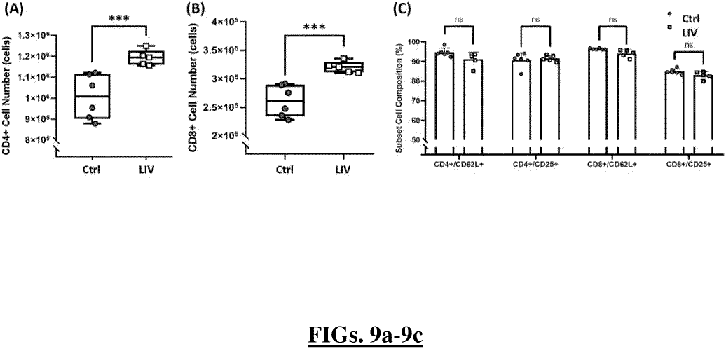

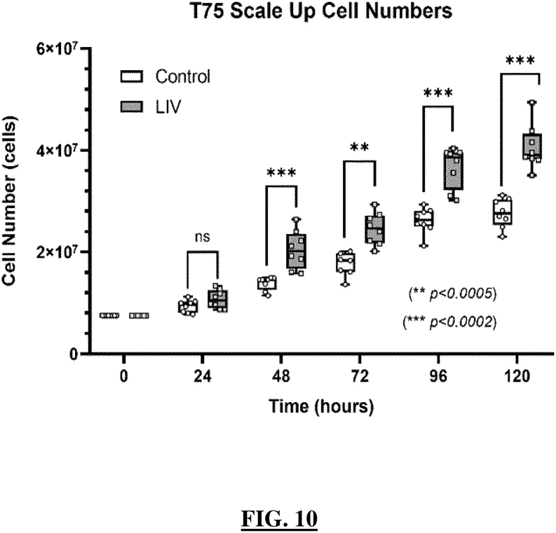

Repeating this signal across 3 bouts further increased proliferation to 39%, emphasizing the importance of daily treatments (FIG. 8b). Flow cytometry was used to investigate the effect of LIV on cell function, evaluating CD4 (helper T cells), CD8 (cytotoxic T cells), CD25 (IL-2 activation), and CD62L (migratory function) biomarkers. While LIV increased both the CD4+ (18.6%; FIG. 9a) and CD8+ (22.6%; FIG. 9b) T cell subpopulations, CD25+/CD62L+ subpopulations were substantially unaltered by LIV signals (FIG. 9c). This indicates that despite the increase in cell number, neither the migratory function nor activation of T cell subsets was altered from exposure to LIV signals. LIV signal exposure can occur to any suitably sized container, from 1 mL or less to many tens, hundreds, or thousands of Liters. One example of such a scale up is shown in FIGS. 10, LIV signal applied to T75 flasks increased proliferation by 50% as compared to controls.

### LIV Signal Changes in Mechanical Environment of Cell Media

Identification of physical mechanism(s) by which cells sense LIV includes the determination of the mechanical milieu enveloping the adherent or suspended cells during exposure. Finite Element Modeling (FEM) has been used to empirically derive LIV-induced fluid shear stresses in vitro, thus, FEM can be used to determine whether LIV signals allowed for separation of two distinct parameters previously proposed to regulate the cellular response to vibration: fluid shear and peak acceleration.

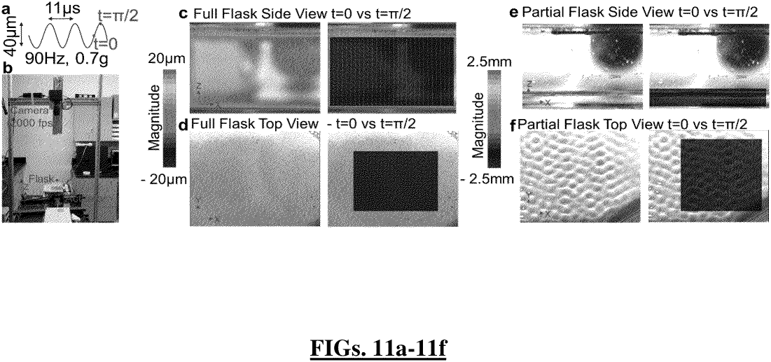

To validate the FEM, Particle Image Velocimetry (PIV) was performed with speckle photography for the measurement of LIV-induced influence on fluid dynamics (FIGS. 11a-11f ). These data established that fluid shear was positively related to peak acceleration magnitude and inversely related to vibration frequency. These data show that shear stress can be separated from acceleration by controlling frequency, acceleration, and/or fluid viscosity for adherent cells, and show good transmission of LIV signals across fluid.

LIV signals applied to an orthogonally, in the embodiments shown in the figures a vertically, oscillating plate (stage) is efficiently and effectively transmitted to non-adherent cells suspended in media. It was tested whether filling a culture vessel maximizes the transmittance of orthogonal accelerations to fluid, the acceleration transmittance in fluid filled, orthogonally oscillating culture vessels was quantified and compared to manufacturer suggested fluid volumes for T75 flasks, as shown in FIGS. 11a-11f.

75 µm speckles with 1 g/mL density were suspended and tracked using a white LED light source, and motion of the surface was captured with a high speed camera (2000 fps; FIGS. 11a and 11b, which is a photograph of the setup). Culture vessels subject to LIV (90 Hz @ 0.7 g via an electrodynamic transducer using a sinusoidal driving function. Fluid motion differential was compared between t=0 and t =π/2, corresponding to the peak cycle displacement of 20 µm. Fluid motion vectors were parallel to each other and to the actuator motion with a maximum magnitude of 18 µm (FIG. 11c), substantially matching the 20 µm peak motion of the actuator, suggesting that fluid particles were accelerating at about the same frequency and acceleration as the actuator. Sloshing at surface was minimal (~2 µm; FIG. 11d), again suggesting minimal fluid motion in filled flasks. In partially filled flasks, displacement vectors peaked at 2.5 mm - two orders of magnitude larger than filled flasks (FIGS. 11e and 11f). Therefore, using filled flasks will minimize fluid sloshing motions and maximize the transmittance of orthogonal LIV.

### LIV Driven Fate-Selection Relies on an Intact Nucleo-Cytoskeleton

Mesenchymal stem cells (MSC) have the capacity to respond to LIV signals by biasing differentiation towards osteoblastogenesis (bone formation) and away from adipogenesis (fat formation). To decipher how mechanical factors influence MSC lineage selection, it has been shown that high magnitude substrate strain (HMS) caused rapid activation of FAK (Focal Adhesion Kinase), an important signaling component of the peripheral focal adhesion, followed by RhoA activation. This signaling pathway, in parallel, inhibits adipocyte differentiation through activation of βcatenin while generating actin restructuring through RhoA.

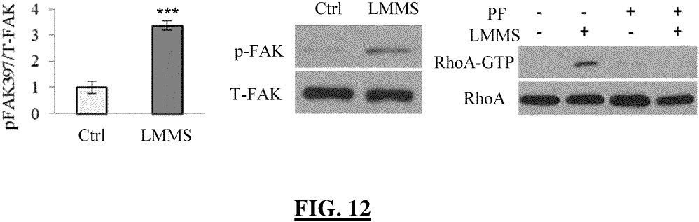

It was then then determined if LIV signals would invoke intracellular signaling pathways in MSC similar to those which occur for HMS (e.g., 0.2 Hz, > 20,000 µε). LIV (20 min @ 90 Hz), a signal which generated <10 µε in the substrate, resulted in FAK phosphorylation, as seen in FIG. 12, followed by FAK-dependent RhoA activation. These data suggest LIV generated signals through the cell cytoskeleton where FAK was located, despite using signals 1/2000^{th} the intensity of stretch.

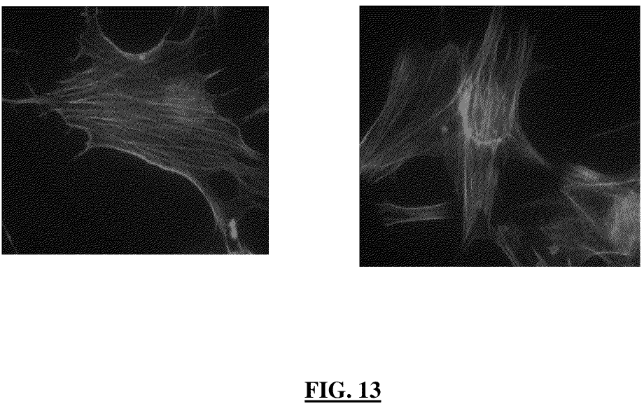

One hour after LIV, F-actin remodeling was concentrated at the perinuclear domain, suggesting the presence of force at the boundary of the nucleus and cytoplasmic cytoskeleton, as shown in the images of FIG. 13. The enhanced actin network suggested that an LIV-adapted cell might serve to prime the cell’s sensitivity to subsequent mechanical signals. As such, synergistic increase of FAK signaling by LIV suggests a pathway for signal amplification of cell responses by “priming” the cell population to LIV.

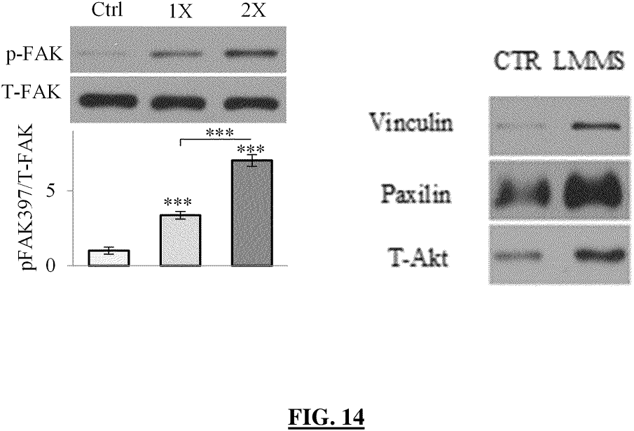

It was then determined if the FAK activation by LIV and downstream activation of RhoA would be amplified with a repeat LIV bout. The second bout of LIV amplified p-FAK response, the results of which are shown in FIG. 14, enhanced formation of focal adhesions. These data indicate that LIV-induced cellular connectivity improves coupling of the nucleus to the plasma edge of the cytoskeleton, and increases overall sensitivity to subsequent mechanical signals: repeat challenges priming the adaptive capacity of the cell to respond to downstream mechanical challenges. These data reinforce the supposition that LINC connectivity of the nucleus plays an important role in cell mechanosensitivity and the mechanoresponse.

### YAP and β-Catenin Are Mechano-Responsive

β-catenin and Yes-associated Protein (YAP) are both transferred into the nucleus to effect stem cell proliferation and lineage events. β-catenin plays a role in the maintenance of self-renewal and the undifferentiated state of stem cells. It has been shown that β-catenin promotes proliferation and stemness of bone marrow mesenchymal cells, enhancing a panel of genes promoting cell cycling. This effect required β-catenin’s direct binding, within the nucleus, of the EZH2 promoter to regulate EZH2 expression and activity. EZH2, which stimulates histone methylation of genes, is anti-differentiative, promoting proliferation. It has been shown in MSC that a two times daily dose of LIV signals can also activate β-catenin.

Without cytoskeletal connection to the nucleus, β-catenin transfer is impaired - as not only is the mechanical signal amplified through the cytoskeleton, but the transfer through nuclear pores requires that the cytoskeleton be intact. LIV signals can promote proliferation through enhancing this universal pathway. Similarly, YAP is a central responder to mechanical signals, albeit studied with static substrate strain. A central molecule in the hippo pathway, YAP enhances proliferation and tissue growth.

### Differences in Cell Stiffness May Contribute to Distinct Response to LIV Signals

Adherent cells respond differently to LIV signals that those in suspension. Given that the cytoskeleton contributes to the biomechanical properties (e.g., stiffness) of a cell, it is plausible that a “softer” cell may respond differently to LIV than a “stiffer” cell. AFM studies have provided direct evidence that mechanical connections between ECM proteins and actin cytoskeleton indeed exist. The differential stiffness of adherent and suspension cells is, in part, due to their physical attachment, or lack of, to a surface. In attached cells, stiffness increases with development of actin fibers within a cell and vinculin controlling cell-cell and cell-matrix junctions. More focal adhesion complex (FAC) clusters are formed in cells attached to 3D suspended structure compared to cells attached to a 2D flat surface.

### T Cell Activation and Expansion Requires Force Generation

Force plays a role in T cell-mediated immune response. In nature, activation of T cells occurs after contact with antigen presenting cells (APC) to form an immunological synapse leading to T cell clonal proliferation to provide the immune response. T cell activation and expansion can be triggered by external forces applied cyclically, such as forces using an atomic force microscope cantilever coated with anti-CD3 to allow the TCR coupling. These data herein provide further evidence that LIV signals can promote T cell expansion.

### METHODS

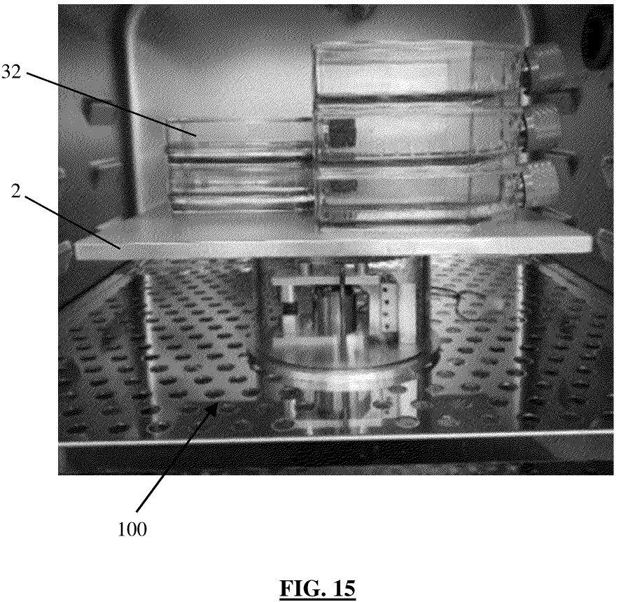

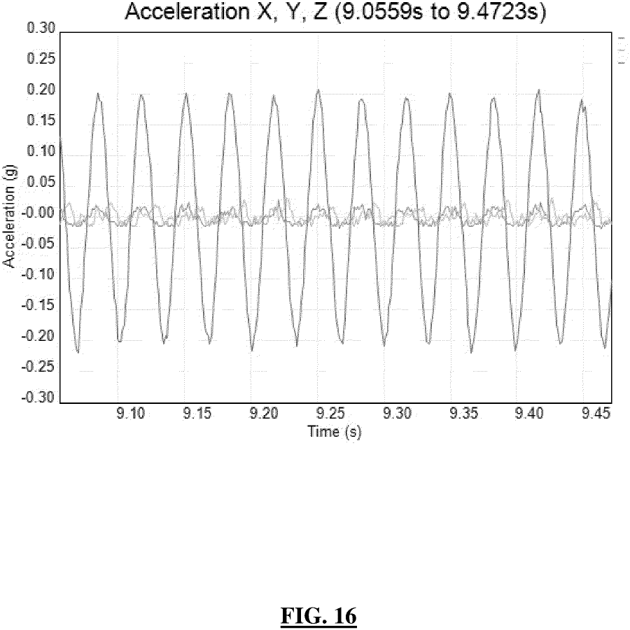

A device 100 is shown in FIG. 15, with a plurality of containers 32 either resting or affixed to the stage 2. As can be seen in FIG. 15, containers 32 can rest, and/or be affixed to one another as well as resting and/or being affixed to the stage 2. The actuator 4 has received a magnitude signal 24 of 0.4 Gₚ₋ₚ, and a frequency signal 22 30 Hz. Movement of the stage 2 is measured by accelerometer 8, the results of which are shown in FIG. 16. FIG. 16 illustrates the control wave, showing high fidelity Z-axis vertical acceleration, with little distortion in the Y or X plane.

### Optimization of Input-Output Relationship

Based on potential applications in biomanufacturing, one focus is on MSC and immune cells (Pan T cells). A partial factorial design defines a series of LIV studies to in terms of frequency, amplitude, duration, dosing and refractory period to maximize cell proliferation (See Table 1). LIV signal parameters can be optimized for MSC and Pan T cell culture in both 24-well plates (SA 1) and T75 flasks (SA 2), for example, but can be cultured in any suitable container. LIV signals can be delivered under sterile conditions and compared to sham across a period of 2, 5 & 10 days, for example.

TABLE 1

LIV Signal Variable

| | | | | | |
|---|---|---|---|---|---|
| Frequency (Hz) | 10 | 30 | 90 | 150 | 500 |
| Intensity (g) | 0.1 | 0.3 | 0.7 | 1.1 | |
| Duration (min) | 5 | 30 | 60 | 120 | |
| Refractory (hours) | - | 1 | 3 | | |
| Doses (x/d) | 1 | 2 | 5 | 10 | |

A partial factorial can be used to define input signal parameters for Pan T cells and MSC. Some examples of such inputs of the LIV signal are shown in Table 1 above.

Outcome measures will be the impact on proliferation at 2, 5 and 10 days. All protocols will be run with n=6, and compared against no-LIV sham control. LINC and internal signaling will be examined at 1,6 & 24 h (S.A. 1b) with ‘effective’ LIV.

### Affect of LIV Signals on Viability and Function of Cells

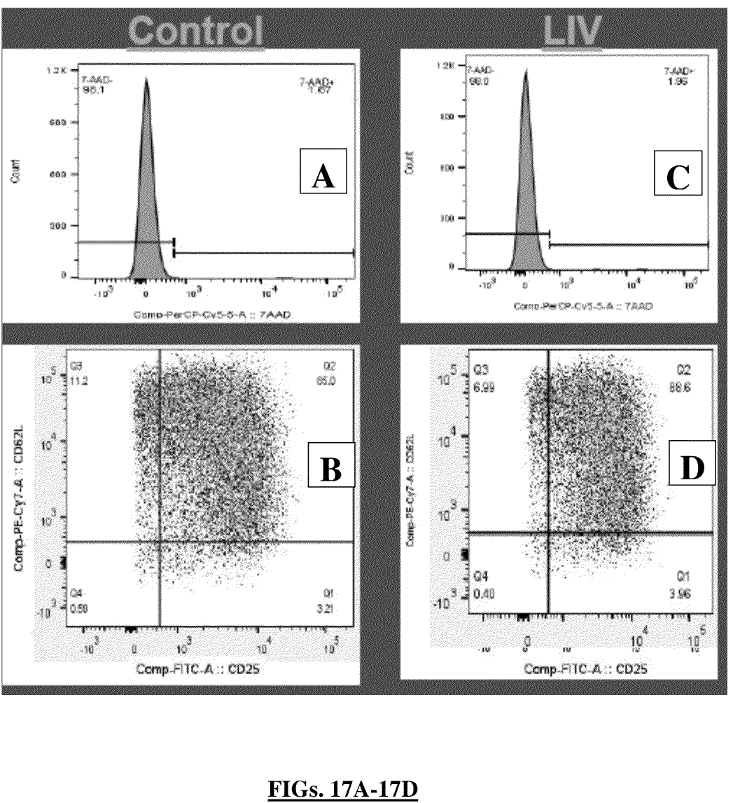

To determine the impact of LIV signals on viability of cells an experiment was conducted with CD4+ T cells. Flow cytometry results are shown in FIGS. 17A-17D are graphical illustrations of the viability and function of CD4+ T cells that have or have not received an LIV signal. The CD4+T cells of FIGS. 17A and 17B did not receive any LIV signal, while the CD4+T cells of FIGS. 17C and 17D received, over 48 hours, a 30 Hz, 0.7 G LIV signal, with a 2 hour refractory period. As can be seen by a comparison between 17A and 17C, the viability of the control cells (7-AAD- cells indicate live cells, 7-ADD+ cells indicate dead cells) as compared to the LIV signal cells indicates that the LIV signal created little or no impact on viability. Similarly, FIGS. 17B and 17D are illustrations of CD25 expression (which is commonly used to check T cell activation function) on the x-axis and CD62L expression (which is commonly used to check T cell mobility function) on the y-axis. As can be seen by a comparison between 17B and 17D, n=4, p=0.82, indicating no significant difference between control cells and LIV signal cells.

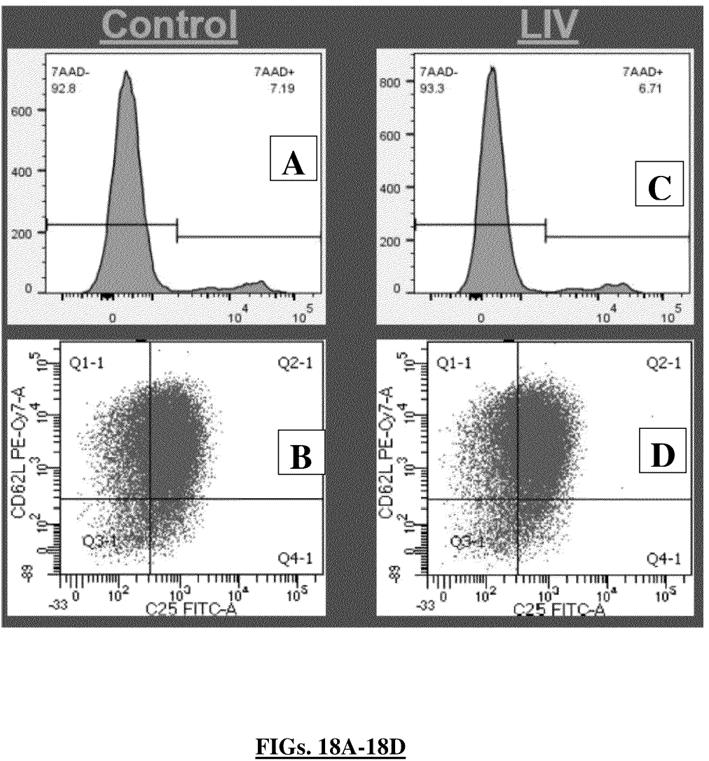

To determine the impact of LIV signals on viability of cells an experiment was conducted with Pan T cells. Flow cytometry results are shown in FIGS. 18A-18D are graphical illustrations of the viability and function of Pan T cells that have or have not received an LIV signal. The Pan T cells of FIGS. 18A and 18B did not receive any LIV signal, while the Pan T cells of FIGS. 18C and 18D received, over 48 hours, a 30 Hz, 0.7 G LIV signal, with a 2 hour refractory period. As can be seen by a comparison between 18A and 18C, the viability of the control cells (~92.8%) as compared to the LIV signal cells (∼93.3%) indicates that the LIV signal created little or no impact on viability (n=5). Similarly, FIGS. 18B and 18D are illustrations of CD25 expression (which is commonly used to check T cell activation function) on the x-axis and CD62L expression (which is commonly used to check T cell mobility function) on the y-axis. As can be seen by a comparison between 18B and 18D, n=4, p=0.13, indicating no significant difference (~2.84%) between control cells and LIV signal cells.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A device comprising:
a stage; and
an actuator configured to transmit a orthogonal force to the stage, wherein the actuator is configured to receive a plurality of orthogonal acceleration signals, wherein the orthogonal acceleration signals comprise an actuator frequency signal and an actuator magnitude signal.

2. The device of claim 1, wherein the actuator frequency signal is between about 0.1 Hz to about 1,000 Hz, or about 10 Hz to about 500 Hz, or about 20 Hz to about 150 Hz, or about 30 Hz to about 90 Hz, or about 30 Hz to about 35 Hz.

3. The device of claim 1, wherein the actuator magnitude signal is about 2 G’s or less, about 1.5 G’s or less, about 1.4 G’s or less, about 1.3 G’s or less, about 1.2 G’s or less, about 1.1 G’s or less, about 1 G or less, about 0.9 G’s or less, about 0.8 G’s or less, about 0.7 G’s or less, about 0.6 G’s or less, about 0.5 G’s or less, about 0.4 G’s or less, about 0.3 G’s or less, about 0.2 G’s or less, or about 0.1 G’s or less.

4. The device of claim 1, wherein the orthogonal acceleration signal further comprises one or more of a duration signal, a refractory period signal and a doses per time signal.

5. The device of claim 1, further comprising an accelerometer operably attached to the stage, wherein the accelerometer is configured to measure at least one of a stage frequency signal and a stage magnitude signal.

6. The device of claim 5, further comprising a processor configured to receive the stage frequency signal and the stage magnitude signal and configured to compare the stage frequency signal to the actuator frequency signal and configured to compare the stage magnitude signal to the actuator magnitude signal.

7. The device of claim 6, wherein the processor is further configured, if there is a difference between the stage frequency signal and the actuator frequency signal, and/or there is a difference between the stage magnitude signal and the actuator magnitude signal, to use the actuator frequency signal and/or the actuator magnitude signal as an input to a machine learning model that predicts whether or not a change to the plurality of orthogonal acceleration signals is to be made.

8. The device of claim 7, wherein the processor is further configured to transmit an updated actuator frequency signal and/or an updated actuator magnitude signal to the actuator based on the predicted change.

9. The device of claim 8, wherein the processor is further configured to access historical data and using the historical data as additional input to the machine learning model.

10. The device of claim 6, wherein if there is a difference between the stage frequency signal and the actuator frequency signal, the processor is configured to automatically transmit an updated actuator frequency signal to the actuator, and wherein if there is a difference between the stage magnitude signal and the actuator magnitude signal, the processor is configured to automatically transmit an updated actuator magnitude signal to the actuator.

11. The device of claim 1, further comprising a container supported by the stage, wherein the container comprises a liquid and a plurality of cells.

12. The device of claim 11, wherein the plurality of cells are selected from stem cells, T cells and combinations thereof.

13. The device of claim 12, wherein the stem cells are mesenchymal stem cells (MSCs).

14. The device of claim 12, wherein the T cells are selected from the group consisting of CD4+ T cells, CD8+ T cells, and CD3+ Pan T cells.

15. The device of claim 11, wherein the plurality of cells are suspended in the liquid, adhered to a surface, or both suspended in the liquid and adhered to the surface.

16. The device of claim 15, wherein the surface comprises a two-dimensional surface or a three-dimensional surface, wherein the two-dimensional surface or the three-dimensional surface is selected from the group consisting of an internal surface of the container, a particle within the container, and combinations thereof.

17. The device of claim 11, further comprising a processor configured to receive an updated concentration of the plurality of cells in the liquid at a time after the actuator receives a plurality of orthogonal acceleration signals and compare the received, updated concentration of the plurality of cells to a goal concentration of the plurality of cells.

18. The device of claim 17, wherein the processor is further configured if there is a difference between the updated concentration of the plurality of cells and the goal concentration, to use the plurality of orthogonal acceleration signals as an input to a machine learning model that predicts whether or not a change to the plurality of orthogonal acceleration signals is to be made.

19. The device of claim 18, wherein the processor is further configured to transmit one or more of an updated actuator frequency signal, an actuator magnitude signal, an updated duration signal, an updated refractory period signal and an updated doses per time signal to the actuator based on the predicted change.

20. The device of claim 19, wherein the processor is further configured to access historical data and using the historical data as additional input to the machine learning model.

21. A method of proliferating cells, the method comprising:
contacting a stage of a device with a container, wherein the device comprises the stage and an actuator configured to transmit a orthogonal force to the stage, and wherein the container comprises a liquid and a plurality of cells;
applying a orthogonal force from the actuator to the stage, wherein the actuator is configured to receive a plurality of the orthogonal acceleration signals, wherein the orthogonal acceleration signals comprise an actuator frequency signal and an actuator magnitude signal.

22. The method of claim 21, wherein the actuator frequency signal is between about 0.1 Hz to about 1,000 Hz, or about 10 Hz to about 500 Hz, or about 20 Hz to about 150 Hz, or about 30 Hz to about 90 Hz, or about 30 Hz to about 35 Hz.

23. The method of claim 21, wherein the actuator magnitude signal is about 2 G’s or less, about 1.5 G’s or less, about 1.4 G’s or less, about 1.3 G’s or less, about 1.2 G’s or less, about 1.1 G’s or less, about 1 G or less, about 0.9 G’s or less, about 0.8 G’s or less, about 0.7 G’s or less, about 0.6 G’s or less, about 0.5 G’s or less, about 0.4 G’s or less, about 0.3 G’s or less, about 0.2 G’s or less, or about 0.1 G’s or less.

24. The method of claim 21, wherein the orthogonal acceleration signal further comprises one or more of a duration signal, a refractory period signal and a doses per time signal.

25. The method of claim 21, wherein the device further comprises an accelerometer operably attached to the stage, wherein, during the applying the orthogonal force from the actuator to the stage step, the accelerometer measures at least one of a stage frequency signal and a stage magnitude signal.

26. The method of claim 25, wherein, during the applying the orthogonal force from the actuator to the stage step, the stage frequency signal and the stage magnitude signal are transmitted, and wherein the method further comprises the step of comparing the stage frequency signal to the actuator frequency signal and the step of comparing the stage magnitude signal to the actuator magnitude signal.

27. The method of claim 26, wherein if there is a difference between the stage frequency signal and the actuator frequency signal, and/or there is a difference between the stage magnitude signal and the actuator magnitude signal, the method further comprises the step of using the actuator frequency signal and/or the actuator magnitude signal as an input to a machine learning model that predicts whether or not a change to the plurality of orthogonal acceleration signals is to be made.

28. The method of claim 27, wherein the method further comprises transmitting an updated actuator frequency signal and/or an updated actuator magnitude signal to the actuator based on the predicted change.

29. The method of claim 28, wherein the method further comprises a step of accessing historical data and using the historical data as additional input to the machine learning model.

30. The method of claim 26, wherein if there is a difference between the stage frequency signal and the actuator frequency signal, the method further comprises automatically transmitting an updated actuator frequency signal to the actuator, and wherein if there is a difference between the stage magnitude signal and the actuator magnitude signal, the method further comprises automatically transmitting an updated actuator magnitude signal to the actuator.

31. The method of claim 21, wherein the plurality of cells are selected from stem cells, T cells and combinations thereof.

32. The method of claim 31, wherein the stem cells are mesenchymal stem cells (MSCs).

33. The method of claim 31, wherein the T cells are selected from the group consisting of CD4+ T cells, CD8+ T cells, and CD3+ Pan T cells.

34. The method of claim 21, wherein the plurality of cells are suspended in the liquid, adhered to a surface, or both suspended in the liquid and adhered to the surface.

35. The method of claim 34, wherein the surface comprises a two dimensional surface or a three dimensional surface, wherein the two dimensional surface or the three dimensional surface is selected from the group consisting of an internal surface of the container, a particle within the container, and combinations thereof.

36. The method of claim 21, wherein the method further comprises that step of receiving an updated concentration of the plurality of cells in the liquid at a time after the applying the orthogonal force from the actuator to the stage step, and the method further comprises the step of comparing the received, updated concentration of the plurality of cells to a goal concentration of the plurality of cells.

37. The method of claim 36, wherein the method further comprises inputting the plurality of orthogonal acceleration signals to a machine learning model that predicts whether or not a change to the plurality of orthogonal acceleration signals is to be made if there is a difference between the updated concentration of the plurality of cells and the goal concentration, to use.

38. The method of claim 37, wherein the method further comprises transmitting one or more of an updated actuator frequency signal, an actuator magnitude signal, an updated duration signal, an updated refractory period signal and an updated doses per time signal to the actuator based on the predicted change.

39. The method of claim 38, wherein the method further comprises accessing historical data and using the historical data as additional input to the machine learning model.

40. A system comprising:
a stage;
an actuator configured to transmit a orthogonal force to the stage, wherein the actuator is configured to receive a plurality of orthogonal acceleration signals, wherein the orthogonal acceleration signals comprise an actuator frequency signal and an actuator magnitude signal; and
a processor configured to receive the stage frequency signal and the stage magnitude signal and configured to compare the stage frequency signal to the actuator frequency signal and configured to compare the stage magnitude signal to the actuator magnitude signal.

41. The system of claim 40, wherein the actuator frequency signal is between about 0.1 Hz to about 1,000 Hz, or about 10 Hz to about 500 Hz, or about 20 Hz to about 150 Hz, or about 30 Hz to about 90 Hz, or about 30 Hz to about 35 Hz.

42. The system of claim 40, wherein the actuator magnitude signal is about 2 G’s or less, about 1.5 G’s or less, about 1.4 G’s or less, about 1.3 G’s or less, about 1.2 G’s or less, about 1.1 G’s or less, about 1 G or less, about 0.9 G’s or less, about 0.8 G’s or less, about 0.7 G’s or less, about 0.6 G’s or less, about 0.5 G’s or less, about 0.4 G’s or less, about 0.3 G’s or less, about 0.2 G’s or less, or about 0.1 G’s or less.

43. The system of claim 40, wherein the orthogonal acceleration signal further comprises one or more of a duration signal, a refractory period signal and a doses per time signal.

44. The system of claim 40, further comprising an accelerometer operably attached to the stage, wherein the accelerometer is configured to measure at least one of a stage frequency signal and a stage magnitude signal.

45. The system of claim 44, wherein the processor is further configured, if there is a difference between the stage frequency signal and the actuator frequency signal, and/or there is a difference between the stage magnitude signal and the actuator magnitude signal, to use the actuator frequency signal and/or the actuator magnitude signal as an input to a machine learning model that predicts whether or not a change to the plurality of orthogonal acceleration signals is to be made.

46. The system of claim 45, wherein the processor is further configured to transmit an updated actuator frequency signal and/or an updated actuator magnitude signal to the actuator based on the predicted change.

47. The system of claim 46, wherein the processor is further configured to access historical data and using the historical data as additional input to the machine learning model.

48. The system of claim 44, wherein if there is a difference between the stage frequency signal and the actuator frequency signal, the processor is configured to automatically transmit an updated actuator frequency signal to the actuator, and wherein if there is a difference between the stage magnitude signal and the actuator magnitude signal, the processor is configured to automatically transmit an updated actuator magnitude signal to the actuator.

49. The system of claim 40, further comprising a container supported by the stage, wherein the container comprises a liquid and a plurality of cells.

50. The system of claim 49, wherein the plurality of cells are selected from stem cells, T cells and combinations thereof.

51. The system of claim 50, wherein the stem cells are mesenchymal stem cells (MSCs).

52. The system of claim 50, wherein the T cells are selected from the group consisting of CD4+ T cells, CD8+ T cells, and CD3+ Pan T cells.

53. The system of claim 49, wherein the plurality of cells are suspended in the liquid, adhered to a surface, or both suspended in the liquid and adhered to the surface.

54. The system of claim 53, wherein the surface comprises a two-dimensional surface or a three-dimensional surface, wherein the two-dimensional surface or the three-dimensional surface is selected from the group consisting of an internal surface of the container, a particle within the container, and combinations thereof.

55. The system of claim 40, wherein the processor is further configured to receive an updated concentration of the plurality of cells in the liquid at a time after the actuator receives a plurality of orthogonal acceleration signals and compare the received, updated concentration of the plurality of cells to a goal concentration of the plurality of cells.

56. The system of claim 55, wherein the processor is further configured if there is a difference between the updated concentration of the plurality of cells and the goal concentration, to use the plurality of orthogonal acceleration signals as an input to a machine learning model that predicts whether or not a change to the plurality of orthogonal acceleration signals is to be made.

57. The system of claim 56, wherein the processor is further configured to transmit one or more of an updated actuator frequency signal, an actuator magnitude signal, an updated duration signal, an updated refractory period signal and an updated doses per time signal to the actuator based on the predicted change.

58. The system of claim 57, wherein the processor is further configured to access historical data and using the historical data as additional input to the machine learning model.
